(19) United States
(12) Patent Application Publication
(10) Pub. No.: US 2019/0043605 A1
(43) Pub. Date: Feb. 7, 2019

(54) Systems and Methods for Analyzing Health Care Data to Improve Billing and Decision Processes

(71) Applicant: RDF Technology, LLC, New Orleans, LA (US)

(72) Inventors: Molly Hegarty, New Orleans, LA (US); Brett Long, New Orleans, LA (US)

(21) Appl. No.: 15/966,749

(22) Filed: Apr. 30, 2018

Related U.S. Application Data
(60) Provisional application No. 62/492,147, filed on Apr. 29, 2017.
(60) Provisional application No. 62/583,336, filed on Nov. 8, 2017.

(51) Int. Cl. G16H 10/60; A61B 5/00; A61B 5/22

(52) U.S. Cl. CPC G16H 10/60; A61B 5/4842; A61B 5/4866; A61B 5/4519; A61B 5/4875; A61B 5/225; A61B 5/4833; A61B 5/4872

(57) ABSTRACT

A method is described herein comprising providing an electronic interface for collecting health care data, wherein the electronic interface integrates with one or more data collection applications of a provider. The method includes receiving malnutrition data of at least one patient through the electronic interface, wherein the electronic interface comprises at least one question relating to malnutrition of the at least one patient. The method includes assessing a malnutrition score for the at least one patient through assessment of the malnutrition data. The method includes adding a malnutrition problem to one or more electronic medical records of the at least one patient when the malnutrition score exceeds a threshold, the adding the problem including updating data intake workflow of the one or more data collection applications to present a malnutrition progress note of the at least one patient to at least one authorized party.

### RELATED APPLICATIONS

This application claims the benefit of U.S. Application No. 62/492,147, Apr. 29, 2017. This application claims the benefit of U.S. Application No. 62/583,336, Nov. 8, 2017.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### BRIEF DESCRIPTION OF THE DRAWINGS

FIG. 1A shows the RDnote application running on a mobile device, under an embodiment.

FIG. 1B shows an RDnote summary data screen, under an embodiment.

FIG. 2 shows a questionnaire screen format, under an embodiment.

FIG. 3 shows a patient intake interface, under an embodiment.

FIG. 4 shows data received through use of RDnote, under an embodiment.

FIG. 5 shows a patient intake interface, under an embodiment.

FIG. 6 shows a patient intake interface, under an embodiment.

FIG. 7 shows a patient intake interface, under an embodiment.

FIG. 8 shows a patient intake interface, under an embodiment.

FIG. 9 shows a patient intake interface, under an embodiment.

FIG. 10 shows a patient intake interface, under an embodiment.

FIG. 11 shows a patient intake interface, under an embodiment.

FIG. 12 shows an RDnote workflow, under an embodiment.

FIG. 13 provides an example of an discharge plan, under an embodiment.

FIG. 14 shows a method for processing malnutrition data, under an embodiment.

### DETAILED DESCRIPTION

The systems and methods described herein (i.e., RDnote technology) involves the use of customized electronic interfaces to collect health care data from consumers of health care. The collected data may then be analyzed on an individual consumer basis or across aggregate sets of consumers to generate information for health care providers that improves billing and health care decision processes.

RDnote technology may use a web application to collect health care data but embodiments are not so limited. The web application may comprise an HTML application or mobile device application.

A clinician working for a healthcare provider may use the RDnote mobile application (or RDnote HTML application) to input patient data based on a survey whose questions RDnote generates on a patient-by-patient basis. Based on sociodemographic data (gender, ethnicity, age, place of residence, etc.) and socioeconomic data (education, income, etc.) obtained either from an (Electronic Medical Record) EMR or from questions in the RDnote software, and/or from information on the patient's clinical condition (e.g., a Congestive Heart Failure or COPD patient), the RDnote software generates questions under an embodiment. These data are transferred to the RDnote API and into the healthcare provider's EMR using a custom and unique RDnote integration. What makes the integration unique is the dynamic questionnaire generation and the synthesis of disparate pieces of existing EMR data for clinical decision support. The data are processed in the RDnote API and database, and the custom EMR integration provides a custom data visualization for the EMR user (clinician).

As just one example with reference to Cerner™, RDnote may provide an “MPage” i.e., an additional page within Cerner™ used by clinicians to administer questions tailored to the patient's condition. An MPage is a Cerner™ medical record software customizable real-time HTML display which RDnote may program to display RDnote data within the Cerner™ medical record software customized to a particular patient or group of patients. These displays allow input from clinicians and can incorporate a variety of medical information about patients. An MPage displays certain selected information both from RDnote data and data synthesized from the EMR and other sources of patient information (other apps, etc.).

Patient data entered into an RDnote interface is deidentified and stored in an RDnote database. RDnote may then perform analytics via algorithms and business logic under one embodiment. All information in RDnote's database including analytics output is de-identified.

RDnote may then conduct original data analysis using these data, and RDnote may send, via its API, three types of analysis to an EMR: billing analytics, clinical analytics, and aggregate-level patient risk analytics for executives.

The RDnote API may use business logic to promote interoperability between different EMR systems, third party APIs, and RDnote's integration with these API. Use of the RDnote API may allow the RDnote API to send and receive patient data from the RDnote application to and from all major EMR systems. We have implemented a unique data model to aggregate key pieces of information from multiple data sources and types. Each of the EMRs has different coding languages and data formats. Having this data model allows RDnote to pull these different types of data, reorganize and analyze the data, and push it back into the medical record. RDnote may aggregate all of its data and run system wide algorithms and business logic on such aggregate (and deidentified) data.

RDnote may provide analytics to hospital executives, update its questions and algorithms based on input from the de-identified data of thousands of patients, and use these analytics to drive system-level improvements across the system of the health provider. RDnote's back-end collects deidentified aggregate socioeconomic, sociodemographic, and clinical data on the patients analyzed by the software. Based on the clinical outcomes noted and the comparative impacts of various interventions, RDnote creates new questions to further refine the quality of data obtained and outcomes achieved.

A general use case scenario for RDnote is provided below.

A healthcare provider may use RDnote software to address one or more of the following problems concerning suboptimal data collection: either 1) the provider fails to capture important data on the procedures and actions of its clinicians to ensure patient health and well-being, and/or 2) the provider captures these data but fails to distribute it to the relevant sections of its EMR or distribute it to the relevant departments to ensure that the provider classifies the patient's condition properly and bills health payers accordingly, and/or 3) the provider distributes the data to the proper entities (billing, doctors, etc.) but end-users fail to utilize these data in a manner that will ensure that the provider classifies the patient's condition properly and bills payers accordingly.

To address one or more of these three problems, providers may offer RDnote software for patients to use in answering clinical questions. Clinicians may input patient data into the RDnote system, and the questionnaires are dynamic, which improves data capture and eliminates duplicative data entry processes. This process improves data capture over existing methods in two ways. First, it eliminates duplicative data entry: presently several clinicians (dietitians, nurses, doctors, etc.) administer the same set of questions to the patient multiple times. RDnote's dynamic questionnaire eliminates this. Second, the questionnaires administered by these clinicians ask several unnecessary questions which lengthen the amount of time spent on surveying. RDnote's dynamic surveys focus only on those questions needed to provide decision support in order for the physician to generate the proper diagnosis. The software then analyzes the de-identified data and generates new data/analytics based on algorithms, business logic, etc. Patient data and RDnote analysis (in the form of billing and clinical documentation) returns to the EMR under one embodiment, and the provider may use clinical documentation data for improved care via RDnote's clinical decision support and billing data to generate additional revenue through improved physician documentation.

Questionnaires are questions that comprise a combination of standard questions embedded into a Cerner™ MPage™ or Powerform™ and dynamic questions delivered from RDnote API via RDnote specific business logic based on inputs from clinicians to Cerner Mpage.

RDnote monitors this process and uses de-identified data gathered from thousands of patients to generate predictive analytics and recommendations for system-wide improvements and analytics for C-suite executives.

Data enters the RDnote environment through patent/clinician interaction under one embodiment. A healthcare worker may administer a questionnaire to patients to obtain data on the patient's activities at home or at a healthcare facility. The questions asked of the patient may be determined based on analysis of existing patient EHR data including biometric, socioeconomic, environmental and medical (i.e., clinical risk factors) information.

As one example, a case manager asks the patient questions to obtain clinically relevant nutrition data.

RDnote software then uses its algorithms and business logic under an embodiment to generate analysis based on the patient's responses. An example includes identifying characteristics of malnutrition after analysis of RD (Registered Dietician) and RN (Registered Nurse) screening and assessment. This analysis generates documentation and clinical decision support for physicians

For example, RDnote aggregates data generated from the web app and uses machine learning and a neural network to predict patient outcomes and risk factors including but not limited to readmission risk, length of stay, severity of illness, risk of mortality, and case mix index. Example input data may include socioeconomic, sociodemographic, and clinical data. Analysis may comprise taking population-level statistics on any number of nutrition-influenced diseases (diabetes, COPD, CHF, etc.) and recommending changes in workflow, non-inpatient interventions, all to improve quality of patient care and reduce the cost to the hospital in the long run. Under an embodiment, RDnote matches a combination of care and risk models to aggregate data on patient profiles and to identify risks of a given patients with similar characteristics. This may provide lower cost for hospitals, higher quality metrics for hospitals, and greater revenue per patient (in some cases) for hospitals.

RDnote may then send this analysis into the health provider's EMR as a) billing documentation for billing departments to generate revenue and b) documentation for clinicians to improve quality of care.

For example, RDnote may provide clinical decision support (CDS) for nutrition related diagnosis and recommended treatments for specific disease states including malnutrition and chronic heart failure.

Upon aggregating sufficient data across a wide sample of a provider's patients, RDnote generates under an embodiment system-wide predictive analytics for hospital management to inform hospital workflow and care procedures.

For example, RDnote may aggregate data generated from the webapp and use machine learning and a neural network to predict patient outcomes and risk factors including but not limited to readmission risk, length of stay, severity of illness, risk of mortality, and case mix index. A hospital system could, for instance, use these data to influence its readmission risk scores (a metric that influences clinician procedures) and optimize clinicians' workflow.

RDnote may use the data and analytics derived from aggregate data analysis to update the questions presented to patients in collecting patient data.

In summary: Clinical questionnaire with questions generated by RDnote according to patient's specifics→RDnote analysis→Analysis results divided into billing information and clinical information→Billing uses analysis to inform the bill; clinicians use analysis to improve quality of care via workflow changes→RDnote analyzes aggregate data to improve the analysis given to billing and clinical departments; this aggregate data analysis creates value for hospital executives by helping them improve quality scores.

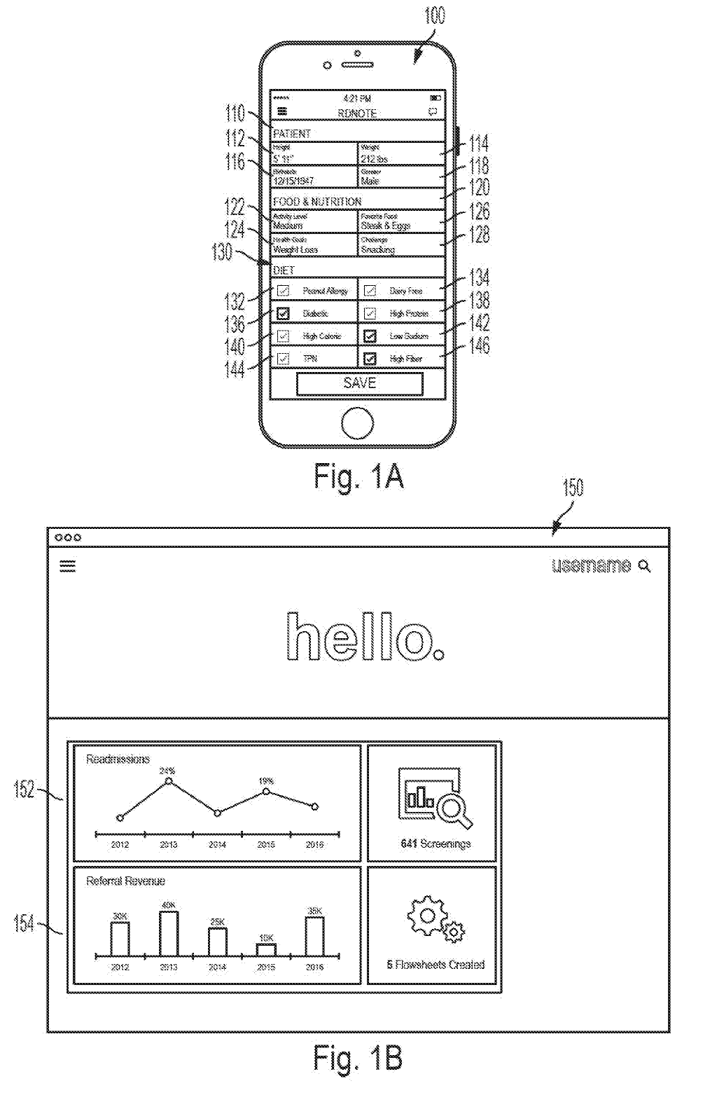

FIG. 1A shows an RDnote application running on a mobile device. The application provides an interface for collecting patient data. For example, the screen 100 of FIG. 1A shows patient data 110 including height 112, weight 114, birthdate 116 and gender 118. Note that the screen displays this patient data in discrete sections, e.g. height—5′11″. However, each visual block displaying a particular piece of information may also provide a touchscreen path to another application screen (not shown) for entering height data and then returning to main screen 100.

The screen of FIG. 1A also shows food and nutrition data 120 including activity level 122, health goal 124, favorite food 126, and challenges 128. Note that the screen displays data in discrete sections, e.g. activity level—medium. However, each visual block displaying a particular piece of information may also provide a touchscreen path to another application screen (not shown) for entering food and nutrition data and then returning to main screen 100.

The screen of FIG. 1A also shows diet data 130 in a check the box format. A user may touch/press one or more displayed diet facts to visually check such fact with a check mark. These facts include peanut allergy 132, dairy free 134, diabetic 136, high protein 138, high calorie 140, low sodium 142, TPN 144, high fiber 146. It is clear from the checked items that a user may highlight one or more items at a time.

FIG. 1B shows a hello screen 150. This initial page may provide summary data. For example, screen 150 shows the readmission rate 152 of patients with respect to a provider under an embodiment. The screen also provides a bar graph describing a provider's referral revenue 154 on a yearly basis, under an embodiment. The screen also shows click through capability to retrieve information regarding 641 screenings and 5 flowsheets.

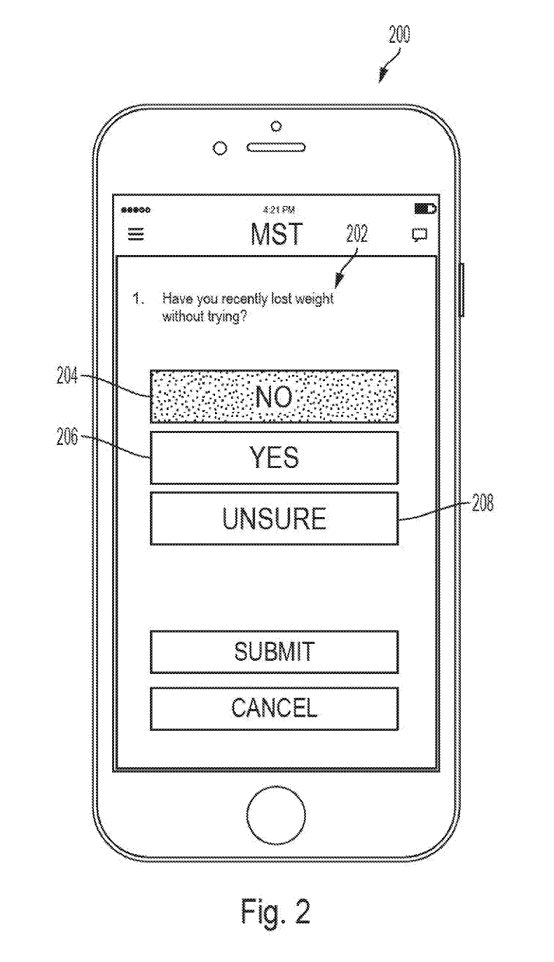

FIG. 2 shows a questionnaire screen format 200. As one example, the questionnaire asks whether a patient has recently lost weight 202 with potential answers no 204, yes 206, and unsure 208. Either patient or provider administering the questionnaire makes a selection and then select submit or cancel.

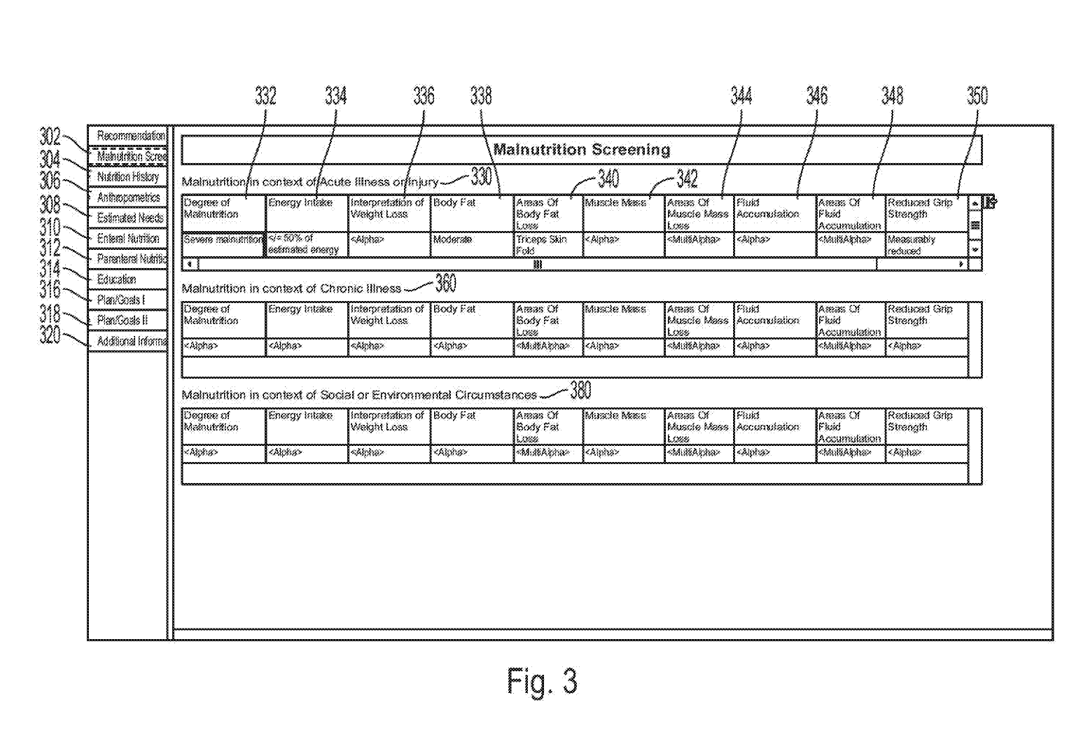

FIGS. 3-8 shows malnutrition clinical decision support interfaces. FIG. 3 comprises an interface for providing malnutrition questions to clinicians, i.e. a malnutrition screen tool (MST). Note that FIGS. 3-8 provide a menu on the upper left side. The menu provides the following options: malnutrition screen 302; nutrition history 304; anthropometrics 306; estimated needs 308; enteral nutrition 310; parenteral nutrition 312; education 314; plan/goals 316; plan/goals 318; additional information 320. Each option provides click through access to separate workflow intakes providing clinicians questions to administer and corresponding input options for data entry.

FIG. 3 shows that the malnutrition screen tab 302 is selected. The malnutrition screening interface provides data intake for “Malnutrition in the Context of Acute Illness or Injury” 330, “Malnutrition in the Context of Chronic Illness” 360, and “Malnutrition in the Context of Social or Environmental Circumstances” 380. Input fields in each row include Degree of Malnutrition 332, Energy Intake 334, Interpretation of Weight Loss 336, Body Fat 338, Areas of Body Fat Loss 340, Muscle Mass 342, Areas of Muscle Mass Loss 344, Fluid Accumulation 346, Areas of Fluid Accumulation 348, and Reduced Grip Strength 350. Each row of data intake comprises fillable fields or click through fields for data entry, i.e. a clinician may click each input option to reach a data entry page for data input.

Note that a minimum of two of the six characteristics above, i.e. negative findings with respect to such characteristics, is recommended for diagnosis of either severe or non-severe malnutrition, under one embodiment. The six characteristics may include energy intake, degree of malnutrition, body fat, muscle mass, fluid accumulation, and reduced grip strength but embodiments are not so limited.

Also note that height and weight should be measured rather than estimated to determine body mass index. Usual weight should be obtained in order to determine the percentage and to interpret the significance of weight loss. Basic indicators of nutritional status such as body weight, weight change, and appetite may substantively improve with refeeding in the absence of inflammation. Refeeding and/or nutrition support may stabilize but not significantly improve nutrition parameters in the presence of inflammation, The National Center for Health Statistics defines “chronic” as a disease/condition lasting 3 months or longer.

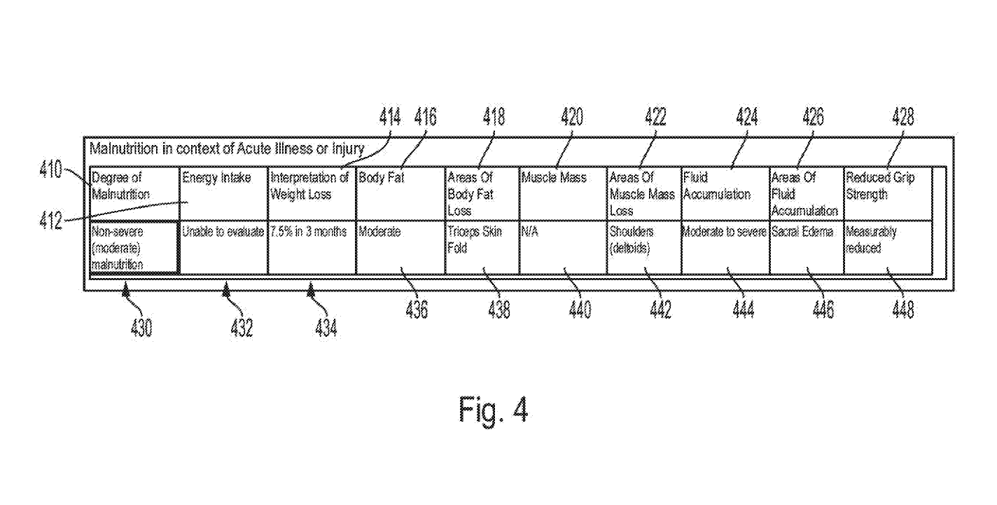

FIG. 4 shows data received through use of RDnote either through questionnaire data and/or clinician input. Data may comprise malnutrition data for patient resulting from acute illness and/or injury as input using the screen of FIG. 3. A health care professional may input such data. A patient may input at least a portion of such data, under an embodiment. Input fields include Degree of Malnutrition 410, Energy Intake 412, Interpretation of Weight Loss 414, Body Fat 416, Areas of Body Fat Loss 418, Muscle Mass 420, Areas of Muscle Mass Loss 422, Fluid Accumulation 424, Areas of Fluid Accumulation 426, and Reduced Grip Strength 428. Corresponding field values include Non-severe (moderate malnutrition) 430, Unable to Evaluate 432, 7.5% in three months 434, Moderate 436, Triceps Skin Fold 438, N/A 440, Shoulders (deltoids) 442, Moderate to Severe 444, Sacral Edema 446, and Measurably reduced 448.

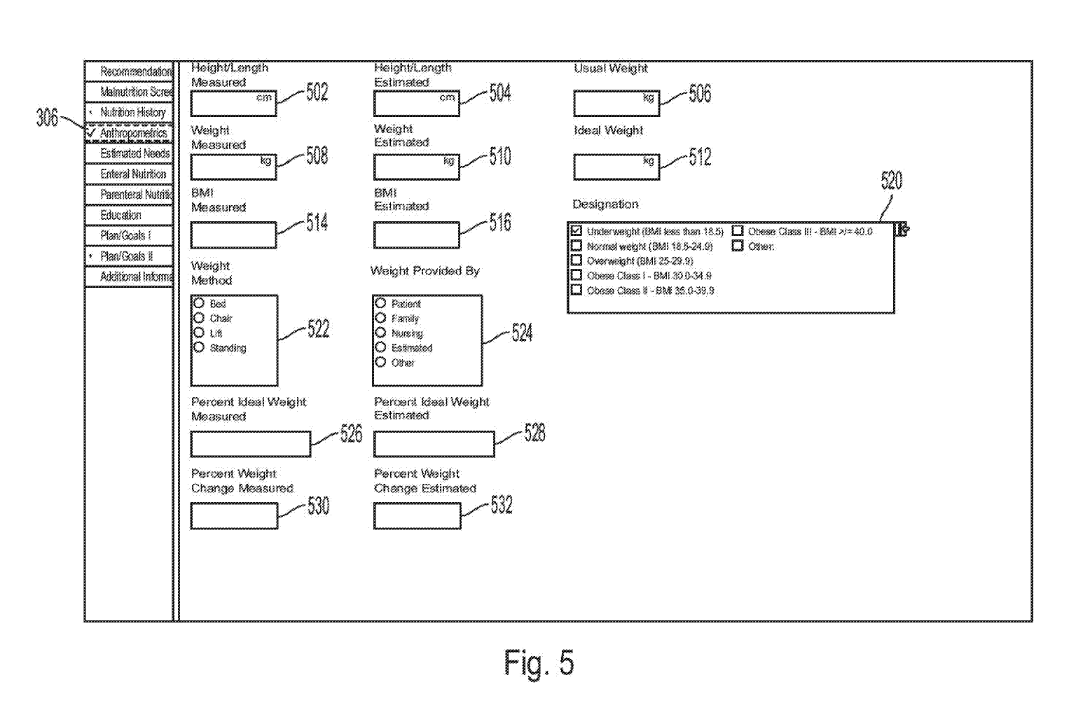

FIG. 5 shows the anthropometrics 309 option selected. FIG. 5 shows the following data input fields: Height/Length measured 502; Height/Length Estimated 504; Usual Weight 506; Weight Measured 508; Weight Estimated 510; Ideal Weight 512; BMI Measured 514; BMI Estimated 516; Designation 520 (including options for underweight (BMI <18.5), normal weight (18.5-24.9), overweight (25-29.9), obese class I (30-34.9); obese class II (35-39.9), obese class III (>/=40.0)).

FIG. 5 shows the following additional input fields: Weight Method 522 including radio button options for bed, chair, lift, standing methods, Weight Provided By 524 including radio button options for patient, family, nursing, estimated options. FIG. 5 also shows the following fields: Percent Ideal Weight Measured 526; Percent Ideal Weight Estimated 528; Percent Weight Change Measured 530; Percent Weight Change Estimated 532.

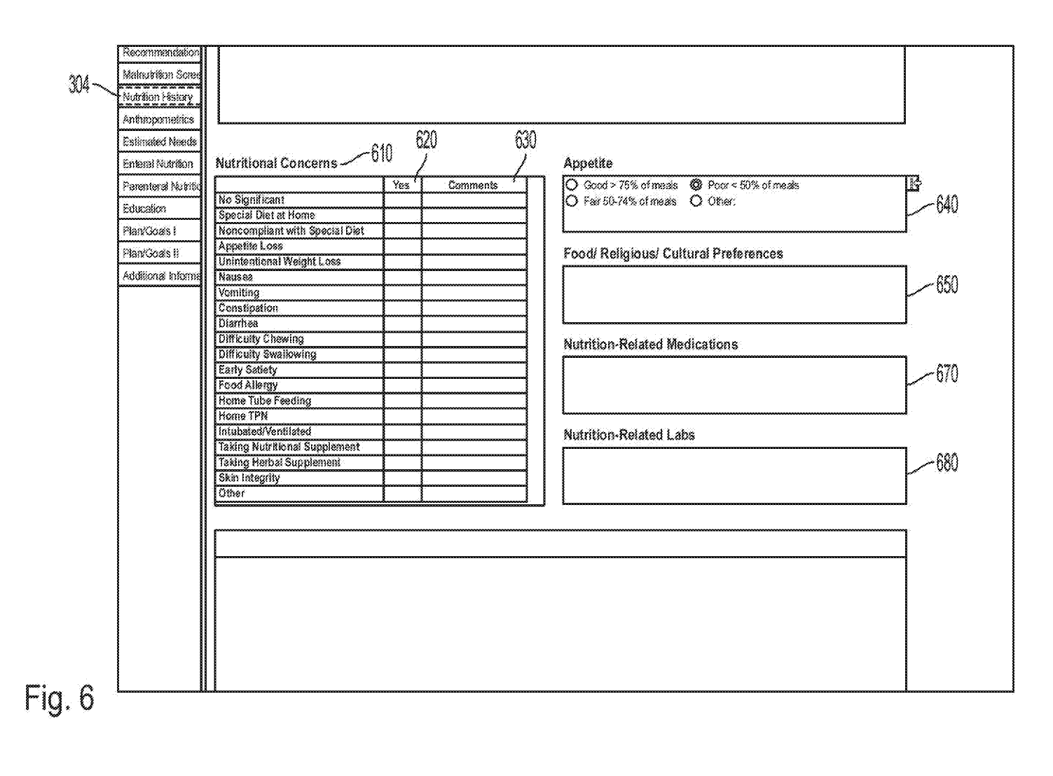

FIG. 6 shows the nutrition history 304 option selected. This screen provides input for various nutritional concerns 610. For example, the clinician may ask a patient whether the patient is experiencing unintentional weight loss. If so, clinician checks a corresponding “Yes” field 620 and may provide free form comments 630. Nutritional concerns 610 include No Significant, Special Diet at Home, Noncompliant with Special Diet, Appetite Loss, Unintentional Weight Loss, Nausea, Vomiting, Constipation, Diarrhea, Difficulty Chewing, Difficulty Swallowing, Early Satiety, Food Allergy, Home Tube Feeding, Home TPN, Intubated/Ventilated, Taking Nutritional Supplement, Taking Herbal Supplement, Skin Integrity, Other. Each nutritional concern allows clinician to check a corresponding “Yes” field 620 and provide free form comments 630. The screen provides additional input for Appetite 640. Clinician may select one of the following corresponding radio buttons to qualify appetite: good >75% of meals, fair 50-74% of meals, poor <50% meals, or other. The screen provides input field 650 for Food/Religious/Cultural preferences. The screen also provides input field 670 for nutrition related medications. The screen also provides input field 680 for nutrition related labs.

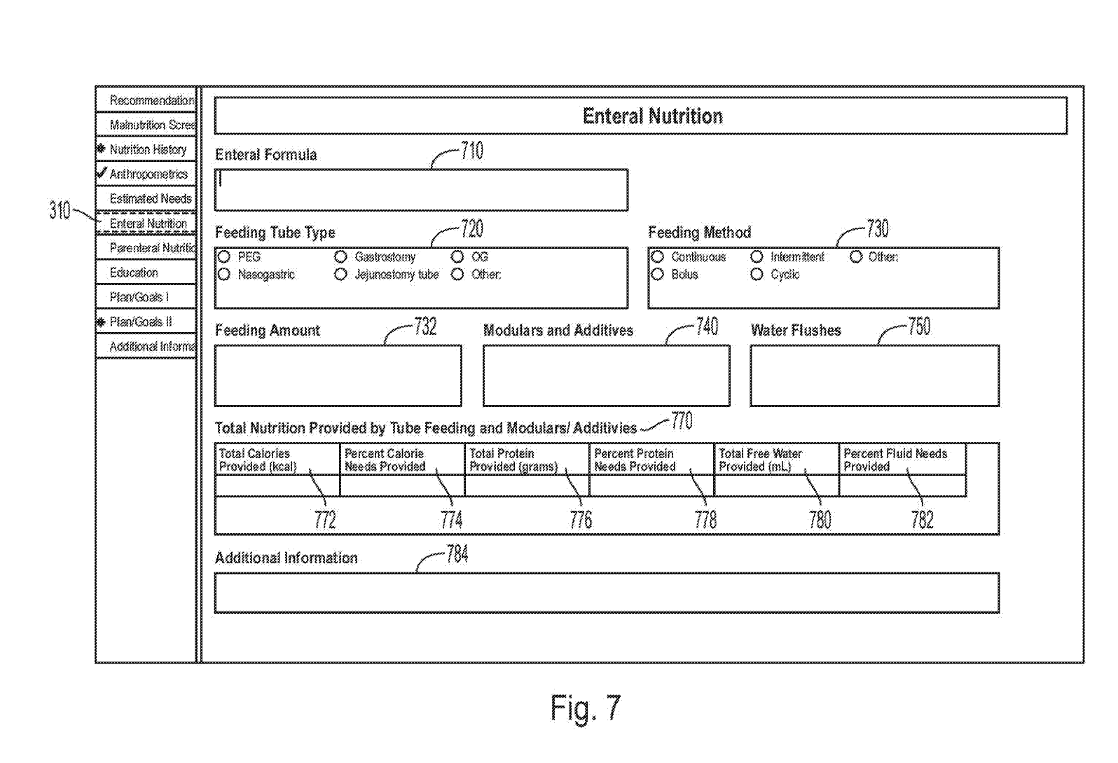

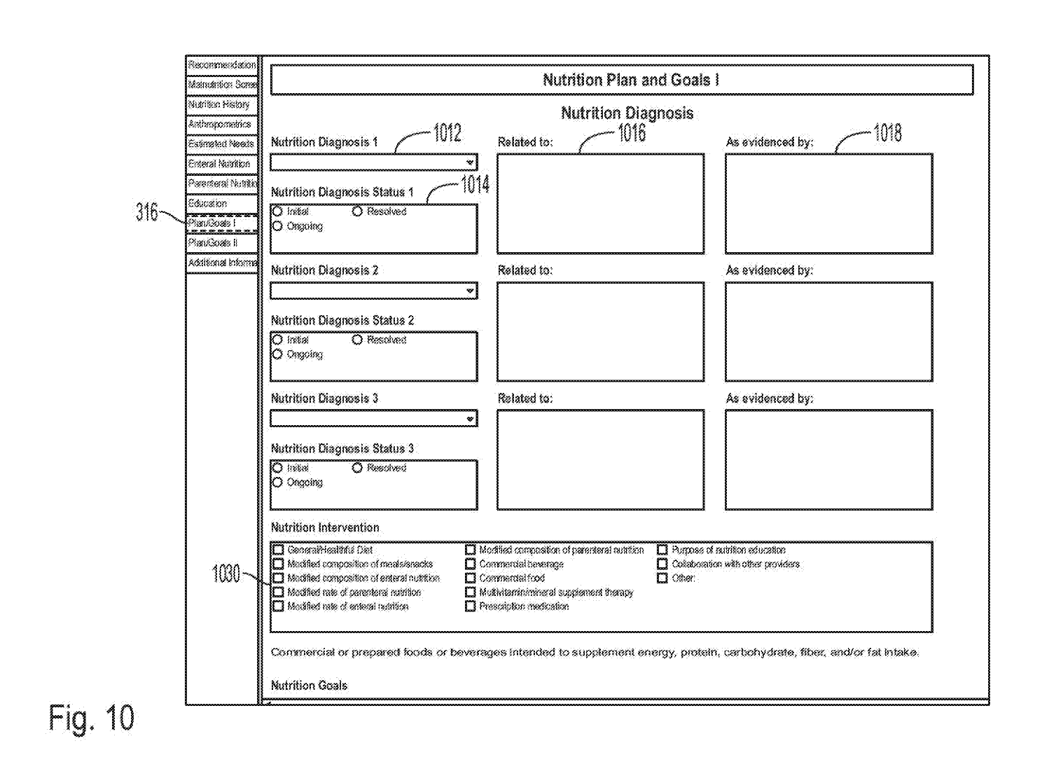

FIG. 7 shows the enteral nutrition 310 option selected. FIG. 7 shows an entry text field for Enteral Formula 710. This figure also provides the following additional input fields: Feeding Tube Type 720 including radio button options for percutaneous endoscopic gastrostomy (PEG), gastrostomy, OG, nasogastric, jejunostomy tube, and other; Feeding Method 730 including radio button options for continuous, intermittent, bolus, cyclic, other. FIG. 10 also provides free form text entry for Feeding Amount 732, Modulars and Additives 740, and Water Flushes 750. The screen shows input options to describe Total Nutrition Provided by Tube Feeding and Modulars/Additives 770. Input options include Total Calories Provided (kcal) 772, Percent Calorie Needs Provided 774, Total Protein Provided (grams) 776, Percent Protein Needs Provided 778, Total Free Water Provided (mL) 780, Percent Fluid Needs Provided 782. The screen then provides free form input option for additional information 784.

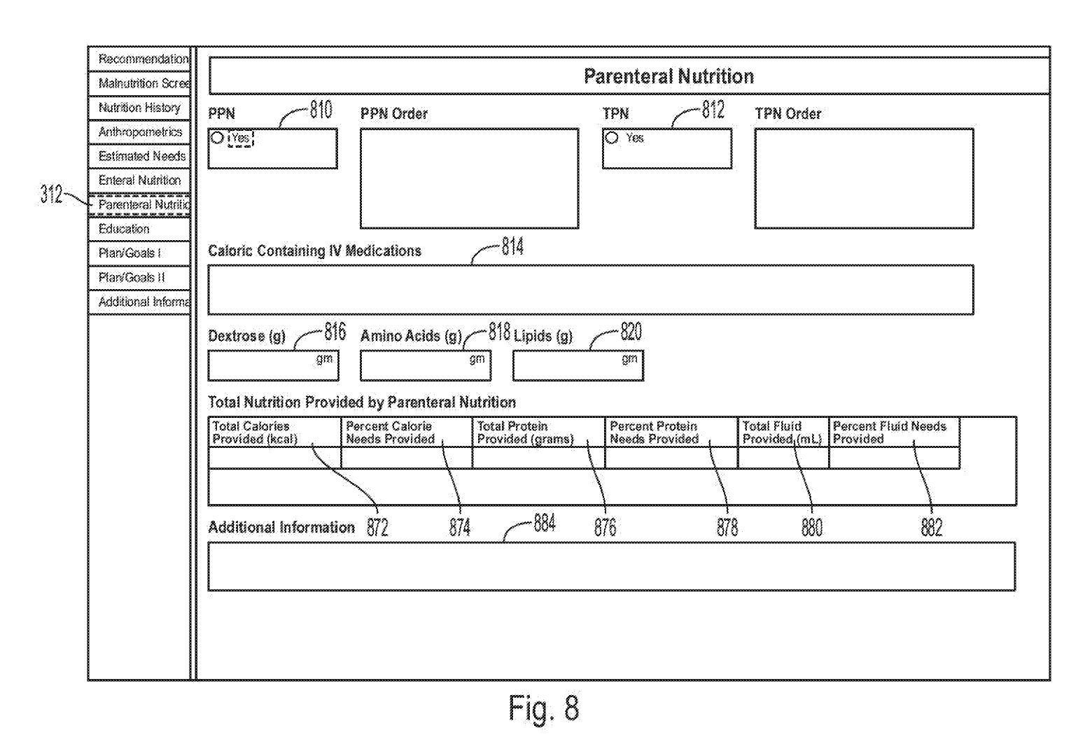

FIG. 8 shows the Parenteral nutrition 312 option selected. FIG. 8 shows a yes radio button option 810 to indicate Peripheral Parenteral Nutrition (PPN). FIG. 8 shows a yes radio button option 812 to indicate Total Parenteral Nutrition (TPN). The screen of FIG. 8 allows the clinician an opportunity to collect input information under the following field entries: Caloric Containing IV Medications 814, Dextrose (g) 816, Amino Acids (g) 818, Lipids (g) 820. The screen shows input options to describe Total Nutrition Provided by Parenteral Nutrition 870. Input options include Total Calories Provided (kcal) 872, Percent Calorie Needs Provided 874, Total Protein Provided (grams) 876, Percent Protein Needs Provided 878, Total Fluid Provided (mL) 880, Percent Fluid Needs Provided 882. The screen then provides free form input option for additional information 884.

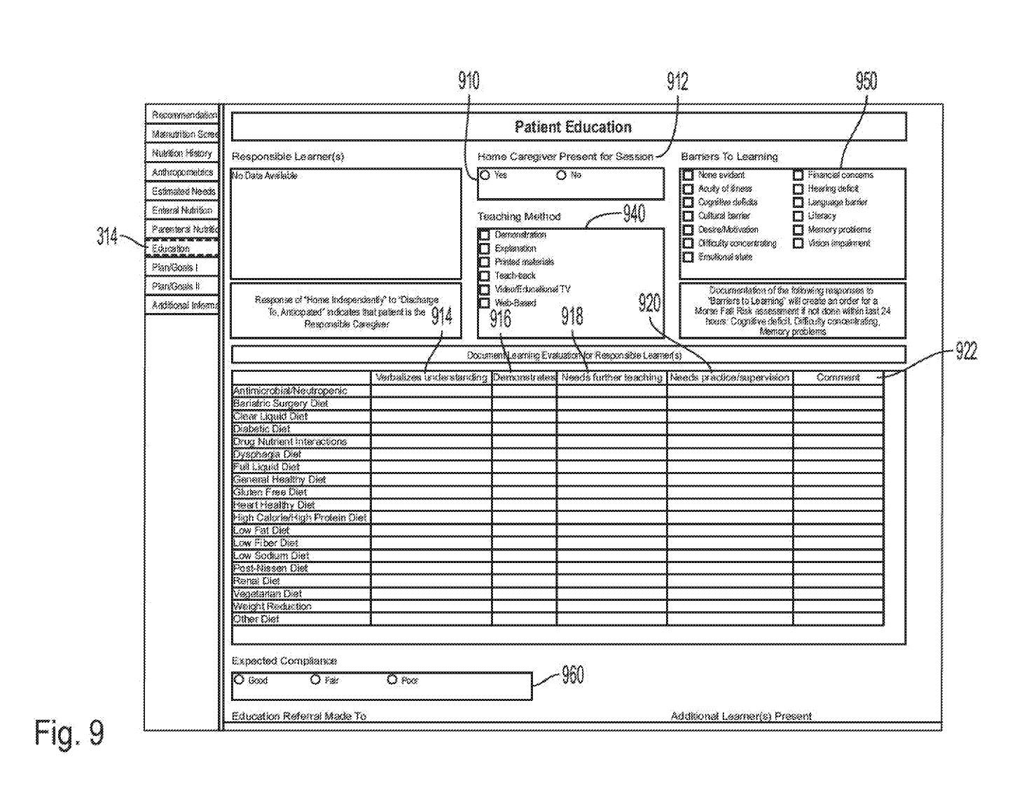

FIG. 9 shows the Education option selected. The screen of FIG. 9 allows clinician to indicate whether a home caregiver is present for a patient education session by checking either a yes or no radio button 910 under a Home Caregiver Present for Session 912 section. Using the screen of FIG. 9, a clinician may evaluate a patient's learning session. The clinician may evaluate the patient's understanding of certain categories of information. For each category, the clinician may indicate that the patient verbalizes understanding 914, demonstrates understanding 916, needs further teaching 918, or needs practice/supervision 920. The clinician may also provide a comment 922 for each category. FIG. 9 provides the following evaluation categories: Antimicrobial/Neutropenic, Bariatric Surgery Diet, Clear Liquid Diet, Diabetic Diet, Drug Nutrient Interactions, Dysphagia Diet, Full Liquid Diet, General Healthy Diet, Gluten Free Diet, Heart Healthy Diet, High Calorie/High Protein Diet, Low Fat Diet, Low Fiber Diet, Low Sodium Diet, Post-Nissen Diet, Renal Diet, Vegetarian Diet, Weight Reduction, Other Diet.

FIG. 9 also solicits Teaching Method 940. The clinician may check on or more of the following options: Demonstration, Explanation, Printed materials, Teach-back, Video/Educational TV, Web-Based. FIG. 10 also requests input regarding Barriers To Learning 950. The clinician may check on or more of the following options: None evident, Acuity of illness, Cognitive deficits, Cultural barrier, Desire/Motivation, Difficulty concentrating, Emotional state, Financial concerns, Hearing deficit, Language barrier, Literacy, Memory problems, Vision impairment. The clinician may rate Expected Compliance 960 with dietary plans using radios buttons for selecting good, fair, or poor.

FIG. 10 shows the Plan/Goals I 316 option selected. The corresponding screen provides a nutrition diagnosis log. For example a clinician may select a nutrition diagnosis topic using drop down menu 1012. The drop down menu provides selection options such as inadequate energy intake, hypercalcemia, etc. The clinician may rate Nutrition Diagnosis Status 1014 using radio buttons to select initial, resolved, or ongoing. The clinician may provide additional information in Related to: 1016 and As evidenced by: 1018 text boxes. A clinician may use the screen of FIG. 10 to indicate one or more nutrition interventions 1030. Interventions may include General/Healthful Diet, Modified composition of meals/snacks, Modified composition of enteral nutrition, Modified rate of parenteral nutrition, Modified rate of enteral nutrition, Modified composition of parenteral nutrition, Commercial beverage, Commercial food, Multivitamin/mineral supplemental therapy, Prescription medication, Purpose of nutrition education, Collaboration with other providers, Other.

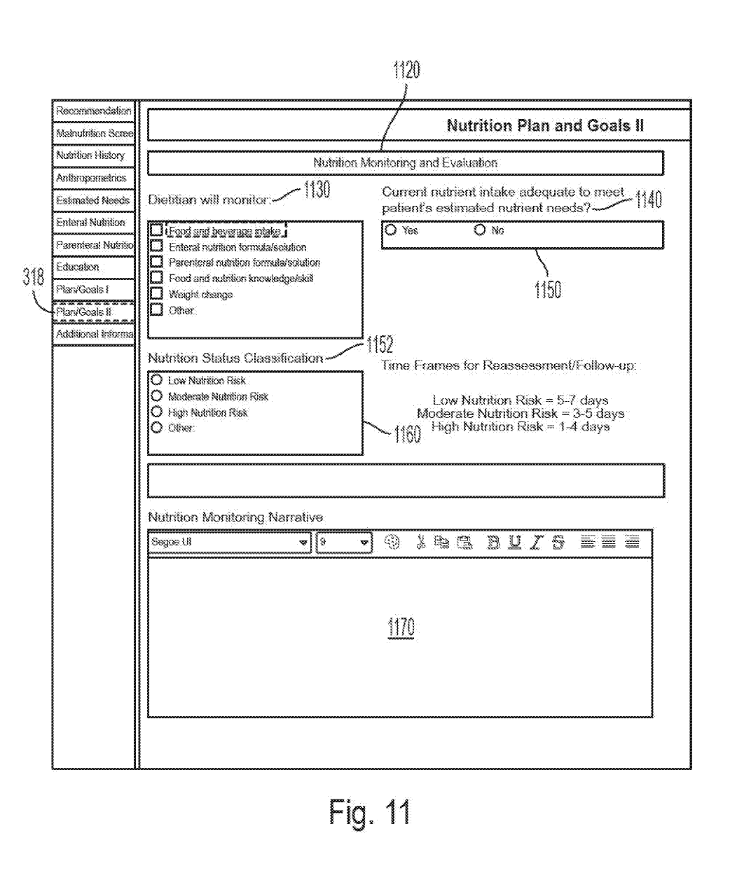

FIG. 11 shows that Plan/Goals II 318 is selected and provides a Nutrition Monitoring and Evaluation 1120 screen. A clinician/dietician may use the interface of FIG. 11 to monitor nutrition status 1130. Monitored status categories include food and beverage intake, enteral nutrition formula/solution, parenteral nutrition formula/solution, food and nutrition knowledge/skill, weight change, and other. A clinician/dietician may indicate whether a patient's current nutrient intake is adequate to meet a patient's estimated medical needs 1140 using yes/no radio buttons 1150. A clinician/dietician may indicate Nutrition Status Classification 1152 using radio buttons 1160 to specify low nutrition risk, moderate nutrition risk, or high nutrition risk. FIG. 11 instructs that reassessment/follow up may be required in 5-7 days for low nutrition risk, 3-5 days for moderate nutrition risk, or 1-4 days for high nutrition risk. FIG. 11 also provides free form text entry for Nutrition Monitoring Narrative 1170 notes.

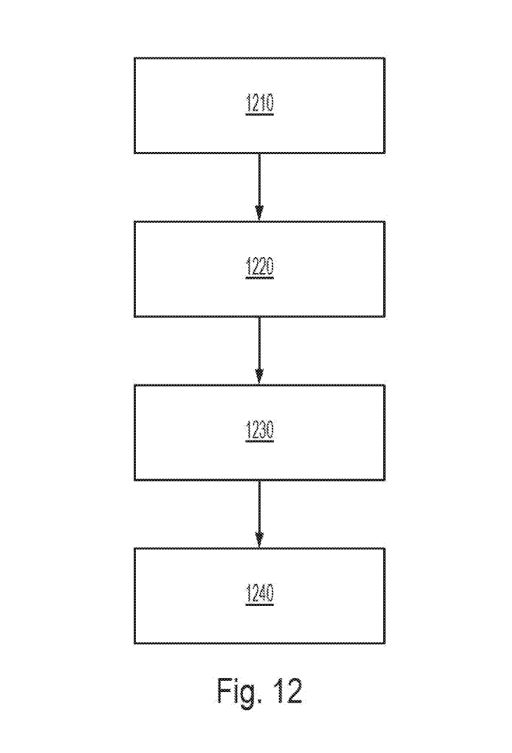

The RDnote platform integrates into standard clinical intake workflows. Under an embodiment as shown in FIG. 12, one or more RDnote applications evaluate a basic adult admit form as step 1210. As part of the admission process, the RDnote malnutrition questionnaire (as seen in FIG. 3) is completed. If the MST score is greater than two, then intake workflow consults an RD who then provides RD/nutrition notes pertaining to the nutrition status of the patient. At step 1220 RDnote pulls MST question responses, MST score, and dosing weight. At step 1230 RDnote evaluates any clinician note regarding nutrition status of patient. If MST score is greater than 2 and/or an alternative threshold negative nutrition status is indicated by the note, then nutrition is added to a problem list in an EMR/EHR as malnutrition mild, moderate, or severe. This creates a hardstop in clinical workflow as described in the next step. At step 1240 a hardstop for a physician is created, i.e. upon opening patient chart a physician is under an embodiment presented with a malnutrition progress note. The malnutrition progress note may be pre-populated with documentation. A physician may comment upon and sign the progress note.

An example of an RD progress note is provided below.

Measurements

Height: 200 cm (Apr. 21, 2017 11:46)

Weight: 73 kg (Apr. 21, 2017) 11:46)

BMI: 18.25 kg/m2 (Apr. 21, 2017) 11:46)

Malnutrition Chronic Illness (04/21/2017 11:46)

Degree of Malnutrition: Severe malnutrition

Energy Intake: <75% of estimated energy requirement for >/=1 month

Interpretation of Weight Loss: 7.5% in 3 months

Body Fat: Severe

Areas of Body Fat Loss: Triceps Skin Fold

Muscle Mass: Mild

Areas of Muscle Mass Loss: Thigh (quadriceps)

Fluid Accumulation: Does not meet criteria

Reduced Grip Strength: Measurably reduced

The RDnote platform performs one or more of the following analytical processes in providing billing and clinical information.

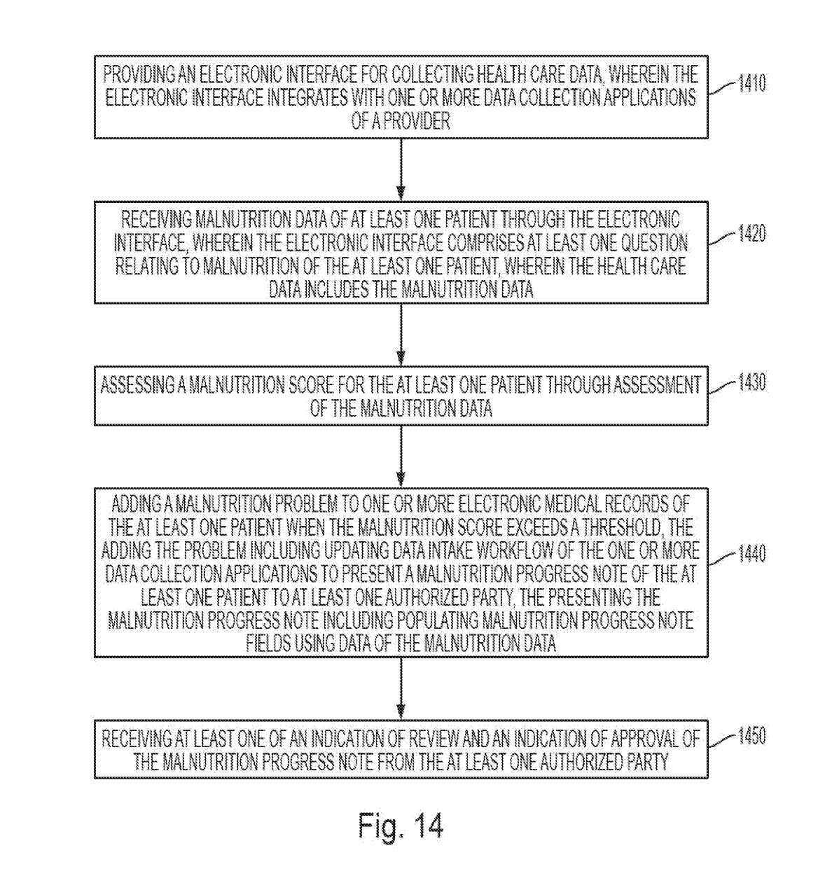

FIG. 14 shows a method for processing malnutrition data. The method includes 1410 providing an electronic interface for collecting health care data, wherein the electronic interface integrates with one or more data collection applications of a provider. The method includes 1420 receiving malnutrition data of at least one patient through the electronic interface, wherein the electronic interface comprises at least one question relating to malnutrition of the at least one patient, wherein the health care data includes the malnutrition data. The method includes 1430 assessing a malnutrition score for the at least one patient through assessment of the malnutrition data. The method includes 1440 adding a malnutrition problem to one or more electronic medical records of the at least one patient when the malnutrition score exceeds a threshold, the adding the problem including updating data intake workflow of the one or more data collection applications to present a malnutrition progress note of the at least one patient to at least one authorized party, the presenting the malnutrition progress note including populating malnutrition progress note fields using data of the malnutrition data. The method includes 1450 receiving at least one of an indication of review and an indication of approval of the malnutrition progress note from the at least one authorized party.

### EXAMPLE ONE

Under one embodiment of an analytical model, RDnote may use input data including (1) existing documentation in hospital (e.g., clinician generated progress notes) and/or (2) clinical and technical workflow data.

RDnote may the use gap analysis, RDnote business logic, and workflow model to provide analytic outputs.

Outputs may include dynamic questions for health care provider to ask in determining malnutrition or other nutrition related disease states. The RDnote software determines under one embodiment the best questions for clinicians to answer to determine whether patient, e.g. Bob, is malnourished and places questions in EMR in a survey format to collect the data from the answers.

Outputs may include dynamic questions for health care provider to ask in determining interventions. RDnote formulates the best questions for clinicians to answer in order to determine whether patient needs nutrition intervention (e.g., supplements) while he/she is at the point of care. RDnote then places these questions in the EMR in a survey form to collect data from the answers.

Outputs may include nutrition intervention and care plan recommendations for health care providers in determining care plans beyond current admission or health care interaction. RDnote software formulates the best questions for clinicians to answer in order to determine whether a patient needs nutrition intervention (e.g., supplements) and support for a certain period beyond current hospitalization and places these care plan and nutrition intervention recommendations in the EMR in a survey format to collect data from the answers. Care plan/Intervention recommendations are generated based on clinical best practice guidelines and RDnote data on effective care plans, under an embodiment.

Outputs may include reports for health care providers regarding billing and compliance. RDnote software formulates the reports for clinicians to assist billing departments with documentation and compliance for patient's diagnosis, intervention, and care plan. Reports are generated by clinician input data that RDnote aggregates and formats. Documentation is reviewed and signed in the EMR by appropriate clinicians, under an embodiment.

### EXAMPLE TWO

Under one embodiment of an analytical model, RDnote may use input data including (1) existing documentation in hospital or clinical setting (e.g., clinician generated progress notes) and/or (2) clinical and technical workflow data.

RDnote may the use gap analysis, RDnote business logic, and/or workflow model to provide analytic outputs.

Outputs may include data to be input into EMR to support diagnosis. RDnote software may under an embodiment analyze the answers from above referenced clinical decision support survey questions and repackage the survey data to place in the EMR in order to support the clinician's decision that patient is malnourished.

Outputs may include data to be input into EMR to support intervention plans. RDnote software may use data gathered from previous RDnote surveys about patient's malnourishment diagnosis to generate suggested tailored clinical interventions for patient. RDnote software then sends data to the EMR recommending best practice nutrition intervention options for malnutrition based on patient's individual needs from previous survey data. The clinician can then recommend an appropriate intervention for patient. For example, a clinician may then choose the intervention under one embodiment of providing Ensure™ to patient three times per day.

Outputs may include data to be input into EMR to support specific care plan. RDnote software uses under an embodiment data gathered from previous RDnote surveys about patient's malnourishment diagnosis and clinician chosen clinical interventions to generate suggested tailored care plan recommendations for patient. RDnote software may send data to the EMR recommending best practice nutrition care plan options for malnutrition based on patient's individual needs from previous survey data. The clinician can then recommend an appropriate care plan for patient. Clinician chooses the care plan of providing Ensure™ to patient three times per day for ninety days.

Outputs may include data to be input into EMR to support billing compliance. RDnote software may under an embodiment use data gathered from previous RDnote surveys about patient's malnourishment diagnosis, clinician chosen clinical interventions and clinician recommended care plans to generate documentation that supports compliance for billing and revenue (i.e. non clinical) purposes. RDnote software may then send data to the EMR based on patient's diagnosis, intervention, and care plan to support the hospital's billing and compliance for patient's diagnosis. Hospital billing and coding departments may use this documentation to appropriately code patient conditions for quality and cost purposes, under an embodiment.

### EXAMPLE THREE

Under one embodiment of an analytical model, RDnote may use input data including (1) data on current hospital diagnosis rates (2) hospital demographics (number of beds, clinicians, etc.), (3) current technical and clinical workflows of hospital, and/or (4) technical and clinical workflows of other hospitals. Technical and clinical workflow data (in this and other examples set forth herein) includes how many and what type of nutritional assessments were made, when, by what clinician type, and diagnosis and/or relevant treatment provided during the patient stay.

RDnote may analyze such input data using data from past projects under an embodiment. RDnote may analyze such input data using clinical & technical workflow analysis. RDnote may create reports and data products including API endpoints, data visualizations, dashboards, etc from the aggregate data. RDnote may analyze such input data using gap analysis.

Output data may include predicted diagnosis rates and clinical efficiency gains if RDnote intervention is implemented. RDnote software may forecast that Hospital X is underdiagnosing malnutrition by a rate of 5% compared to healthcare organizations with similar patient profiles.

Output data may include predicted intervention changes and clinical efficiency gains if an RDnote intervention is implemented. RDnote may forecast that Hospital X would capture 15 percent clinical efficiency improvements by implementing an RDnote clinical intervention planning tool compared to healthcare organizations with similar patient profiles.

Output data may include predicted care plan changes and clinical efficiency gains if an RDnote intervention is implemented. RDnote may forecast that Hospital X would capture 17 percent clinical efficiency improvements by implementing an RDnote care planning tool compared to healthcare organizations with similar patient profiles.

Output data may include predicted billing impact and clinical efficiency gains if an RDnote intervention is implemented. RDnote software may forecast that hospital X may capture 9% more accurate diagnosis, 7% time savings for clinician and medical coder time by implementing an RDnote documentation tool such as the malnutrition documentation intake workflow/questionnaire described above.

### EXAMPLE FOUR

Under one embodiment of an analytical model, RDnote may use input data including (1) data on current hospital diagnosis rates (2) hospital demographics (number of beds, clinicians, etc.), (3) current technical and clinical workflows of hospital, and/or (4) technical and clinical workflows of other hospitals.

RDnote may analyze such input data using data from past projects under an embodiment including patient characteristics, clinician behavior, interventions, care plan compliance, outcomes, etc. as collected by one or more applications of the RDnote platform. RDnote may analyze such input data using clinical & technical workflow analysis RDnote may analyze such input data using gap analysis. Under an embodiment, gap analysis comprises differences between best practices and observed baselines.

Output data may include predicted changes in revenue for a particular disease diagnosis. RDnote software may predict revenue generated by capturing 5% higher diagnosis rate for similarly undiagnosed malnourished patients like patient referenced in this and other examples above, based on analysis of Hospital X's practices and RDnote modeling.

Output data may include predicted changes in revenue for a particular clinical intervention. RDnote may predict revenue generated by capturing 5% higher intervention rate for similarly undiagnosed malnourished patients like patient referenced in this and other examples above, based on analysis of Hospital X's practices and RDnote modeling.

Output data may include predicted changes in revenue for a particular care plan. RDnote may predict revenue generated by capturing 5% higher care plan rate for similarly undiagnosed malnourished patients like patient referenced in this and other examples above, based on analysis of Hospital X's practices and RDnote modeling.

Output data may include predicted change in revenue for a particular documentation change. RDnote may predict 5% increase in revenue generated by capturing proper documentation, for example 10% clinician time saved in charting.

### EXAMPLE FIVE

Under one embodiment of an analytical model, RDnote may use input data including (1) data on current hospital diagnosis rates (2) hospital demographics (number of beds, clinicians, etc.), (3) current technical and clinical workflows of hospital, and/or (4) technical and clinical workflows of other hospitals.

RDnote may analyze such input data using data from past projects under an embodiment. RDnote may analyze such input data using clinical & technical workflow analysis. RDnote may analyze such input data using gap analysis.

Output data may include predicted changes in quality metrics (length of stay, severity of illness, readmission rate, etc.) if certain diagnoses are documented. RDnote may predict changes in quality measures such as Lengthy of Stay (LOS), Space Occupying Lesion (SOL), readmission, etc., if 5% more patients receive a malnutrition diagnosis.

Output data may include predicted changes in quality metrics if certain clinical interventions are introduced at the point of care. RDnote may predict changes in quality measures such as LOS, SOL readmission, etc., if 5% more patients receive a particular intervention for their malnutrition diagnosis.

Output data may include predicted changes in quality metrics if certain care plans are implemented after the patient is discharged. RDnote may predict changes in quality measures such as LOS, SOL readmission, etc., if 5% more patients receive a particular care plan for their malnutrition diagnosis.

Output data may include predicted change in quality metrics if certain billing documentation is implemented. RDnote may model changes in reimbursement related to increased quality measures, such as LOS (Length of Stay), SOI (Severity of Illness), etc. if RDnote tools for diagnosis, clinical intervention, and/or care planning are implemented.

Under one embodiment, the RDnote platform may implement an RDnote connect service. RDnote Connect may provide nutrition discharge planning and compliance tracking for patients leaving a hospital and discharging to their home or to a home-health, skilled nursing, and/or long-term acute care facility. Upon a patient's leaving an inpatient setting, clinicians may develop many plans for each patient regarding compliance with various medical needs, such as medication compliance. For some patients a nutrition plan is created upon discharge. RDnote Connect facilitates creating nutrition discharge plans within the EHR and distributing it electronically to other health providers who are unable to access the hospital's electronic medical record (which is the original repository of the nutrition discharge plan). This facilitates greater compliance with nutrition discharge plans, resulting in lower costs and higher-quality outcomes for patients and health organizations.

A nutrition risk assessment score and discharge plan may be created by an MD based on an RD's recommendations and plan suggestions provided by RDnote's algorithms for a patient upon conclusion of their stay in an inpatient hospital.

The risk assessment may include assessment of the following information: age, admission date, current and working medical diagnosis, medication lists, nutrition questions including nutritional intake, social determinants of health (economic, demographic, etc.), biometrics, the RD's professional assessment of the patient's nutritional status and disease-state specific nutritional conditions.

A nutrition risk score is then generated under an embodiment from the above assessment to alert clinicians to the complexity and risk of the patient's nutritional status. Risk score relates the risk of a patient for increased complications if nutritional status if not addressed. Numerical Risk Scores range from 1-5 and Percentage 0-100%, under an embodiment.

This discharge nutrition care plan may contain nutrition prescriptions, recommendations and specific diet and fluid instructions for the patient to comply with outside of the hospital.

RDnote's algorithms may take data such as patient age, mobility, social determinants of health, biometrics, EHR data and medical treatment and care plans into account to determine a risk score and recommend customized nutrition interventions based on the patient's unique needs. The plan is recommended to the physicians and care team in charge of the patient; the physicians and care team may review, edit, and/or accept the discharge care plan. Risk score may be based on patient demographics, biomentrics, disease states, clinician behavior, etc., under an embodiment.

This discharge nutrition care plan may be stored in a medical record (e.g., Cerner™ or Epic™) which is used by the hospital.

RDnote Connect may under an embodiment securely electronically transfer/send the discharge care plan from hospital medical record to the tablet devices used by outside clinical providers: nurses, physicians etc., who work in outside health clinics including: home health, skilled nursing, or long-term acute care. This may be done using RDnote APIs as well as HL7 messaging.

Health Level-7 or HL7 refers to a set of international standards for transfer of clinical and administrative data between software applications used by various healthcare providers. These standards focus on the application layer, which is “layer 7” in the OSI model. The HL7 standards are produced by the Health Level Seven International, an international standards organization, and are adopted by other standards issuing bodies such as American National Standards Institute and International Organization for Standardization.

Hospitals and other healthcare provider organizations typically have many different computer systems used for everything from billing records to patient tracking. All of these systems should communicate with each other (or “interface”) when they receive new information, or when they wish to retrieve information.

HL7 International specifies a number of flexible standards, guidelines, and methodologies by which various healthcare systems can communicate with each other. Such guidelines or data standards are a set of rules that allow information to be shared and processed in a uniform and consistent manner. These data standards are meant to allow healthcare organizations to easily share clinical information. Theoretically, this ability to exchange information should help to minimize the tendency for medical care to be geographically isolated and highly variable.

Continuing with the example of RDConnect set forth above, clinicians are able to view the nutrition discharge plan and monitor compliance and feed information back into the RDnote Connect application. The RDnote application generates a tracking and compliance plan based on the information in the discharge care plan. The appropriate clinician monitors this plan and tracks patient and provider compliance with the plan details. This information may be securely transferred, updated and stored in the hospital electronic medical record as well.

Clinicians working in the hospital seeking to reduce readmissions into their facilities may monitor the information sent back to the EMR via the RDnote Connect application to determine which patients have the highest risk of readmissions. Where necessary, they may recommend additional interventions to reduce the probability of patients returning to the hospital.

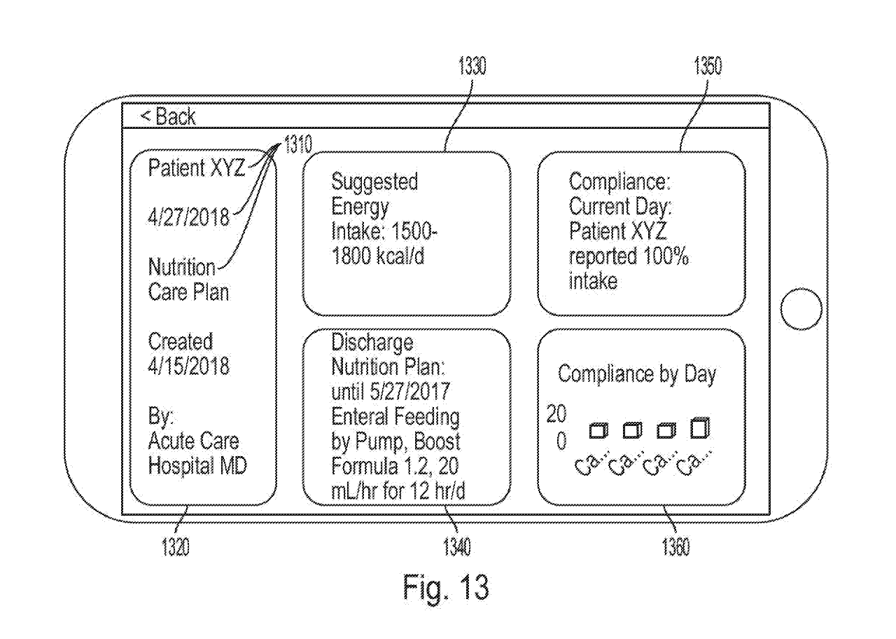

FIG. 13 provides an example of an discharge plan. FIG. 13 shows a patient's discharge nutrition care plan as of Apr. 27, 2018 (1310) created by Acute Care Hospital MD 1320. As shown the plan was created on Apr. 15, 2018. The plan shows 1330 suggested energy intake of 1500-1800 kcal/d. FIG. 13 describes 1340 the discharge nutrition plan through May 27, 2017: enteral feeding by pump, boost formula 1.2, 20 mL/hr for 12 hr/d. FIG. 13 shows compliance 1350 for current day at 100% intake. The patient may use RDnote Connect software to report compliance under an embodiment. FIG. 13 may also show patient compliance by day 1360.

Under one embodiment, the RDnote platform may implement an RDnote Home service. Patients in a home or home health setting could benefit from greater nutrition care, the lack of which makes existing medical problems worse while increasing the cost of that patient's care for the health system. RDnote Home reaches patients within the home by creating customized interventions and tracks compliance with the recommended interventions for patients, tailored to their unique demographic and medical needs. This could range from a text message to check in with patients to an in-house consultation with a Registered Dietitian who could provide intensive one-on-one care. By providing proper care before a patient's conditions worsen, RDnote Home lowers healthcare costs and increases quality of health outcomes, under an embodiment.

RDnote Home may be used by healthcare providers, such as hospitals, and health insurance companies to create plans for patients in a variety of settings. These types of organizations use, respectively, electronic medical records and claims data to monitor their patients. Depending on the client in question, the RDnote Home software works as further described below under an embodiment.

Based on analysis of the electronic medical record data (for hospitals) or claims data (for insurers), RDnote Home creates under an embodiment a risk assessment score for each patient and identifies patients with a high risk of readmissions who could also benefit from a tailored nutrition care program.

The risk assessment may comprise an assessment of the following information: age, admission date, current and working medical diagnosis, medication lists, nutrition questions including nutritional intake, social determinants of health (economic data, demographic statistics, etc.), biometrics, the RD's professional assessment of the patient's nutritional status and disease-state specific nutritional conditions.

RDnote's algorithms take data such as patient age, mobility, social determinants of health, biometrics, EHR data and medical treatment and care plans into account to determine a risk score and recommend customized nutrition interventions based on the patient's unique needs, under an embodiment. The plan is recommended to the physicians and care team in charge of the patient; the physicians and care team may review, edit, and/or accept/decline the home nutrition care plan.

RDnote devises the proper nutrition care intervention for these flagged patients.

RDnote delivers the intervention and monitors its progress. Data includes patient compliance with recommendations, biometrics such as weight, hydration level, medication compliance, exercise, and sleep.

As one example of RDnote Home, RDnote software reviews claims data of a health insurance company and flags a member with gestational diabetes. RDnote Home assigns a nutrition risk score and creates a customized intervention and tracking plan and sends a copy of the plan to the primary care physician via the RDnote API.

RDnote Home delivers these plans and interventions via secure email and text message to the member/patient and tracks member compliance with the plans. The compliance data are tracked and digested into usable data which is then transmitted via RDnote APIs back into the primary care physicians' EHR, under an embodiment.

A method of an embodiment includes an electronic interface for collecting health care data, wherein the electronic interface integrates with one or more data collection applications of a provider. The method includes receiving malnutrition data of at least one patient through the electronic interface, wherein the electronic interface comprises at least one question relating to malnutrition of the at least one patient, wherein the health care data includes the malnutrition data. The method includes assessing a malnutrition score for the at least one patient through assessment of the malnutrition data. The method includes adding a malnutrition problem to one or more electronic medical records of the at least one patient when the malnutrition score exceeds a threshold, the adding the problem including updating data intake workflow of the one or more data collection applications to present a malnutrition progress note of the at least one patient to at least one authorized party, the presenting the malnutrition progress note including populating malnutrition progress note fields using data of the malnutrition data.

The method of an embodiment includes receiving at least one of an indication of review and an indication of approval of the malnutrition progress note from the at least one authorized party.

The method of an embodiment includes using the malnutrition data of the at least one patient to update content of the at least one question.

The malnutrition data of an embodiment comprises data relating to at least one of energy intake, degree of malnutrition, body fat, muscle mass, fluid accumulation, and reduced grip strength.

The method of an embodiment includes using an application programming interface to provide the malnutrition data to the one or more electronic medical records.

The method of an embodiment includes using the malnutrition data to recommend one or more nutrition interventions for the least one patient, wherein the malnutrition data comprises data of the one or more nutrition interventions.

The method of an embodiment includes tracking compliance of the at least one patient with the one or more nutrition interventions, wherein the malnutrition data comprises the compliance data of the at least one patient.

The method of an embodiment includes using the malnutrition data to analyze aggregate health care data of the at least one patient.

A method of an embodiment includes an electronic interface for collecting health care data, wherein the electronic interface integrates with one or more data collection applications of a provider. The method includes receiving malnutrition data of at least one patient through the electronic interface, wherein the electronic interface comprises at least one question relating to malnutrition of the at least one patient, wherein the health care data includes the malnutrition data. The method includes assessing a malnutrition score for the at least one patient through assessment of the malnutrition data. The method includes adding a malnutrition problem to one or more electronic medical records of the at least one patient when the malnutrition score exceeds a threshold, the adding the problem including updating data intake workflow of the one or more data collection applications to present a malnutrition progress note of the at least one patient to at least one authorized party, the presenting the malnutrition progress note including populating malnutrition progress note fields using data of the malnutrition data. The method includes receiving at least one of an indication of review and an indication of approval of the malnutrition progress note from the at least one authorized party.

Computer networks suitable for use with the embodiments described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network may be any microprocessor controlled device that permits access to the network, including terminal devices, such as personal computers, workstations, servers, mini computers, main-frame computers, laptop computers, mobile computers, palm top computers, hand held computers, mobile phones, TV set-top boxes, or combinations thereof. The computer network may include one of more LANs, WANs, Internets, and computers. The computers may serve as servers, clients, or a combination thereof.

The systems and methods for analyzing health care data to improve billing and decision processes can be a component of a single system, multiple systems, and/or geographically separate systems. The systems and methods for analyzing health care data to improve billing and decision processes can also be a subcomponent or subsystem of a single system, multiple systems, and/or geographically separate systems. The components of systems and methods for analyzing health care data to improve billing and decision processes can be coupled to one or more other components (not shown) of a host system or a system coupled to the host system.

One or more components of the systems and methods for analyzing health care data to improve billing and decision processes and/or a corresponding interface, system or application to which the systems and methods for analyzing health care data to improve billing and decision processes is coupled or connected includes and/or runs under and/or in association with a processing system. The processing system includes any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein refers to any logic processing unit, such as one or more central processing units (CPUs), digital signal processors (DSPs), application-specific integrated circuits (ASIC), etc. The processor and memory can be monolithically integrated onto a single chip, distributed among a number of chips or components, and/or provided by some combination of algorithms. The methods described herein can be implemented in one or more of software algorithm(s), programs, firmware, hardware, components, circuitry, in any combination.

The components of any system that include the systems and methods for analyzing health care data to improve billing and decision processes can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the systems and methods for analyzing health care data to improve billing and decision processes and corresponding systems and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the systems and methods for analyzing health care data to improve billing and decision processes and corresponding systems and methods include: microcontrollers with memory (such as electronically erasable programmable read only memory (EEPROM)), embedded microprocessors, firmware, software, etc. Furthermore, aspects of the systems and methods for analyzing health care data to improve billing and decision processes and corresponding systems and methods may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein may be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the above described components may be processed by a processing entity (e.g., one or more processors) within the computer system in conjunction with execution of one or more other computer programs.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import, when used in this application, refer to this application as a whole and not to any particular portions of this application. When the word “or” is used in reference to a list of two or more items, that word covers all of the following interpretations of the word: any of the items in the list, all of the items in the list and any combination of the items in the list.

The above description of embodiments of the systems and methods for analyzing health care data to improve billing and decision processes is not intended to be exhaustive or to limit the systems and methods to the precise forms disclosed. While specific embodiments of, and examples for, the systems and methods for analyzing health care data to improve billing and decision processes and corresponding systems and methods are described herein for illustrative purposes, various equivalent modifications are possible within the scope of the systems and methods, as those skilled in the relevant art will recognize. The teachings of the systems and methods for analyzing health care data to improve billing and decision processes and corresponding systems and methods provided herein can be applied to other systems and methods, not only for the systems and methods described above.

The elements and acts of the various embodiments described above can be combined to provide further embodiments. These and other changes can be made to the systems and methods for analyzing health care data to improve billing and decision processes and corresponding systems and methods in light of the above detailed description.

We claim:

1. A method comprising,
providing an electronic interface for collecting health care data, wherein the electronic interface integrates with one or more data collection applications of a provider;
receiving malnutrition data of at least one patient through the electronic interface, wherein the electronic interface comprises at least one question relating to malnutrition of the at least one patient, wherein the health care data includes the malnutrition data;
assessing a malnutrition score for the at least one patient through assessment of the malnutrition data;
adding a malnutrition problem to one or more electronic medical records of the at least one patient when the malnutrition score exceeds a threshold, the adding the problem including updating data intake workflow of the one or more data collection applications to present a malnutrition progress note of the at least one patient to at least one authorized party, the presenting the malnutrition progress note including populating malnutrition progress note fields using data of the malnutrition data.

2. The method of claim 1, receiving at least one of an indication of review and an indication of approval of the malnutrition progress note from the at least one authorized party.

3. The method of claim 1, comprising using the malnutrition data of the at least one patient to update content of the at least one question.

4. The method of claim 1, wherein the malnutrition data comprises data relating to at least one of energy intake, degree of malnutrition, body fat, muscle mass, fluid accumulation, and reduced grip strength.

5. The method of claim 1, comprising using an application programming interface to provide the malnutrition data to the one or more electronic medical records.

6. The method of claim 1, using the malnutrition data to recommend one or more nutrition interventions for the least one patient, wherein the malnutrition data comprises data of the one or more nutrition interventions.

7. The method of claim 1, comprising tracking compliance of the at least one patient with the one or more nutrition interventions, wherein the malnutrition data comprises the compliance data of the at least one patient.

8. The method of claim 1, comprising using the malnutrition data to analyze aggregate health care data of the at least one patient.

9. A method comprising,
providing an electronic interface for collecting health care data, wherein the electronic interface integrates with one or more data collection applications of a provider;
receiving malnutrition data of at least one patient through the electronic interface, wherein the electronic interface comprises at least one question relating to malnutrition of the at least one patient, wherein the health care data includes the malnutrition data;
assessing a malnutrition score for the at least one patient through assessment of the malnutrition data;
adding a malnutrition problem to one or more electronic medical records of the at least one patient when the malnutrition score exceeds a threshold, the adding the problem including updating data intake workflow of the one or more data collection applications to present a malnutrition progress note of the at least one patient to at least one authorized party, the presenting the malnutrition progress note including populating malnutrition progress note fields using data of the malnutrition data;
receiving at least one of an indication of review and an indication of approval of the malnutrition progress note from the at least one authorized party.
